(19) United States
(12) Patent Application Publication
(10) Pub. No.: US 2020/0330511 A1
(43) Pub. Date: Oct. 22, 2020

(54) Relocation of Stem Cells

(71) Applicant: Clear LLC, Mill Valley, CA (US)

(72) Inventor: Beth McDougall, Mill Valley, CA (US)

(21) Appl. No.: 16/855,614

(22) Filed: Apr. 22, 2020

Related U.S. Application Data
(60) Provisional application No. 62/837,154, filed on Apr. 22, 2019.

(51) Int. Cl. A61K 35/12; C12N 13/00; A61P 25/28; A61P 9/04

(52) U.S. Cl. CPC A61K 35/12; C12N 13/00; A61K 9/0019; A61P 9/04; A61P 25/28

(57) ABSTRACT

A stem cell therapy using an energy field (e.g., a laser beam) to prime stem cells, and then using a matching energy field to direct the primed stem cells to a target tissue. Preferably, the matching energy field has the same frequency as the energy field use to prime the stem cells (with no more than 10 Hz variation). Stem cells can be primed ex vivo or in vivo, by being exposed to the energy field, and/or a growth hormone. The stem cell therapy can be used to treat diseases including heart disease, spinal cord injury, and neurodegenerative diseases.

### PRIORITY INFORMATION

This application claims the benefit of priority to U.S. Provisional Patent Application No. 62/837,154, filed on Apr. 22, 2019, which is incorporated herein by reference in its entirety.

### FIELD OF THE INVENTION

The field of the invention is relocation of stem cells to a target region.

### BACKGROUND

The following description includes information that may be useful in understanding the present invention. It is not an admission that any of the information provided herein is prior art or relevant to the presently claimed invention, or that any publication specifically or implicitly referenced is prior art.

Stem cells are special purpose cells that have the potential to assist with repair and maintenance of the body. Their most critical properties are that they are capable of dividing more or less without limit, and can then either stay in the source pool or leave it to become a replacement cell. Pluripotent stem cells are the most versatile in that they are capable of becoming any type of cells in the body. Other types of stem cells (e.g., multipotent stem cells) are more restricted in what types of cells they can become. The stem cells used in treatment are either autologous (i.e., from the same person) or allogeneic (i.e., from another person).

There are two major limitations to the efficacy of stem cell therapy. The first challenge lies in directing the stems cells to where they are needed. For example, stem cells delivered intravenously tend to circulate with the blood flow and only a small portion of stem cells can ever reach a target tissue where they are needed. Moreover, even when the stem cells reach a target tissue, they may not stay there. For example, stem cell treatments for heart failure, using a catheter to invasively inject stem cells directly into the arteries supplying the heart, often lead to disappointing results because most of the stem cells delivered into the heart likely end up flowing out of the heart instead of staying there. It is only when the stem cells are directed to the desired tissue, and then anchor there, that they can be effectively involved in tissue repair and regeneration.

U.S. Pat. Nos. 9,999,785 and 10,202,598 to Todd Ovokaitys teach a method that uses a laser beam to activate stem cells, and then guides the activated stem cells to a target tissue using a transcutaneous, multi-axis homing beam. . However, since the intersecting volume of laser beams is typically quite small, use of intersecting beams has limited ranges and applications, especially for large tissues and organs.

Thus, there is still a need for a method of directing stem cells to sites of injury in vivo.

All publications identified herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

### SUMMARY OF THE INVENTION

The inventive subject matter provides apparatus, systems and methods in which an energy field (e.g., a laser beam) is used to prime stem cells, and a matching energy field is used to direct the primed stem cells to a target region.

In some embodiments, the stem cells (either autologous or allogeneic) are primed ex vivo by exposing them to a first energy field before they are infused into a patient. The first energy field can include light (e.g., visible light, infrared, ultraviolet light, and laser), plasma radiation, acoustic waves (including ultrasound), nuclear magnetic resonance (NMR), or any combination thereof. The first energy field has an energy signature, including frequency, intensity, waveform, and amplitude, etc. Alternatively or additionally, the stem cells are chemically primed with one or more growth factors (e.g., fibroblast growth factor (FGF), insulin, insulin-like growth factors, vascular endothelial growth factor (VEGF), migration-stimulating factor, etc). The step of chemically priming the stem cells can be performed before, during, or after priming the stem cells by exposing them to the first energy field.

The primed stem cells are then infused into a patient, preferably intravenously. Shortly after priming (e.g., within 10 minutes), the primed stem cells are directed toward a target region of the patient's body by subjecting the target region to a second energy field that matches at least one of the energy signatures of the first energy field used to prime the stem cells. The matched energy signature can be one or more of frequency, intensity, waveform, and amplitude, etc. The underlying theory is that since the stem cells were attuned to the first energy field during the priming process, the primed stem cells develop a “memory” of the first energy field. Afterwards, they migrate toward and stay at the target region exposed to the same or similar energy field. The matching energy field also causes the stem cells to express adhesion molecules (e.g., integrins) that allow the stem cells to anchor down to the target tissue.

In preferred embodiments, the second energy field has a frequency that matches the frequency of the first energy field, with less than 1000 Hz difference. For example, if the first energy field has a frequency of 1,000,000 Hz, then the second energy field has a frequency between of 999,000 and 1,001,000 Hz. More preferably, the second energy field has less than 100 Hz difference in frequency compared to the first energy field. More preferably, the second energy field has less than 10 Hz difference in frequency compared to the first energy field. Even more preferably, the second energy field has less than 1 Hz difference in frequency compared to the first energy field.

In preferred embodiments, the energy field used to prime and to direct the stems cells is a red laser beam. The red laser beam has a power level that is low enough to avoid pain or burning of the skin. In fact, the skin is barely warmed, after a typical duration of 5-15 minutes with the laser beam applied to the target area through the skin. The red laser wave form has several times the depth of penetration through tissue than other types of lasers or visible light that only goes up to 5 millimeters, or about a quarter inch, through the skin. This makes it practical to concentrate stem cells in tissues and organs located more deeply in the body. More preferably, multiple laser beams are directed at an internal volume of tissue from two or more directions, the strongest signal for the cells to localize can be in the precisely desired region where the multiple laser beams intersect.

In one experiment, a laser was directed through a culture flask of cells with stem cell characteristics that were randomly circulating. 24 hours later the cells in the flask had been strongly drawn to and were concentrated in the area the beam had been located. This effect was so strong that the cells were visible to the naked eye as a linear strand of cells. Whereas this cell line may aggregate 8-16 cells together it was estimated that 1-2 million cells were adherent to each other in an organized column. Moreover, exposure to the laser increased integrin expression of these cells by 40-100%.

In contemplated embodiments, autologous stem cells are primed in vivo and then directed to a target region of the patient. In the priming process, at least a portion of the body of the patient is subjected to a first energy field. Preferably, an area of the patient having rich blood supply and/or minimal pigment in the skin is subjected to the first energy field. Contemplated areas include mucous membrane (e.g., nasal cavity, mouth) of the patient, tympanic membrane, nasal cavity, and eye. In some embodiments, a patient's entire body is subjected to the first energy field.

Alternatively or additionally, priming the stem cells in vivo can include injecting a patient with one or more growth factors (e.g., fibroblast growth factor (FGF), insulin, insulin-like growth factors, vascular endothelial growth factor (VEGF), migration-stimulating factor, etc). The step of injecting growth factors can be performed before, during, or after priming the stem cells by exposing the patient to the first energy field.

To direct the primed stem cells to a target region, only the target region of the body is preferably exposed to a second energy field that matches at least one of the energy signatures of the first energy field. Preferably, the second energy field used to direct the stem cells matches the frequency or frequencies of the first energy field that was used to prime the stem cells, where the “matching” means that the difference in at least one frequency is less than 1000 Hz, more preferably less than 100 Hz, still more preferably less than 10 Hz, and even more preferably less than 1 Hz.

The target region in the patient can be any region that needs healing or repairing, including for purposes of anti-aging, rejuvenation, and tissue regeneration. For example, in a patient with spinal cord injury, a preferred target region is the spinal cord. In a patient with neurodegenerative diseases (e.g., Parkinson's, dementia, Alzheimer's, etc.) or stroke, the target region is the brain. In a patient with heart disease (e.g., heart failure), a preferred target region is the heart. Other contemplated diseases or conditions to be treated include orthopedic issues (e.g., osteoarthritis and bone fractures), torn rotator cuffs, degenerative disc disease, endocrine depletion, hair loss, multiple sclerosis, cerebral palsy, peripheral neuropathy, and ALS.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIG. 1 is a flowchart showing steps of priming the stem cells ex vivo with a laser beam, infusing the primed stem cell into a patient, and directing the infused stem cells to a target region of the body.

FIG. 2 is a flowchart showing steps of priming the stem cells in vivo with a laser beam, and directing the primed stem cells to a target region of the body.

### DETAILED DESCRIPTION

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Unless the context dictates the contrary, all ranges set forth herein should be interpreted as being inclusive of their endpoints, and open-ended ranges should be interpreted to include only commercially practical values. Similarly, all lists of values should be considered as inclusive of intermediate values unless the context indicates the contrary.

The recitation of ranges of values herein is merely intended to serve as a shorthand method of referring individually to each separate value falling within the range. Unless otherwise indicated herein, each individual value with a range is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

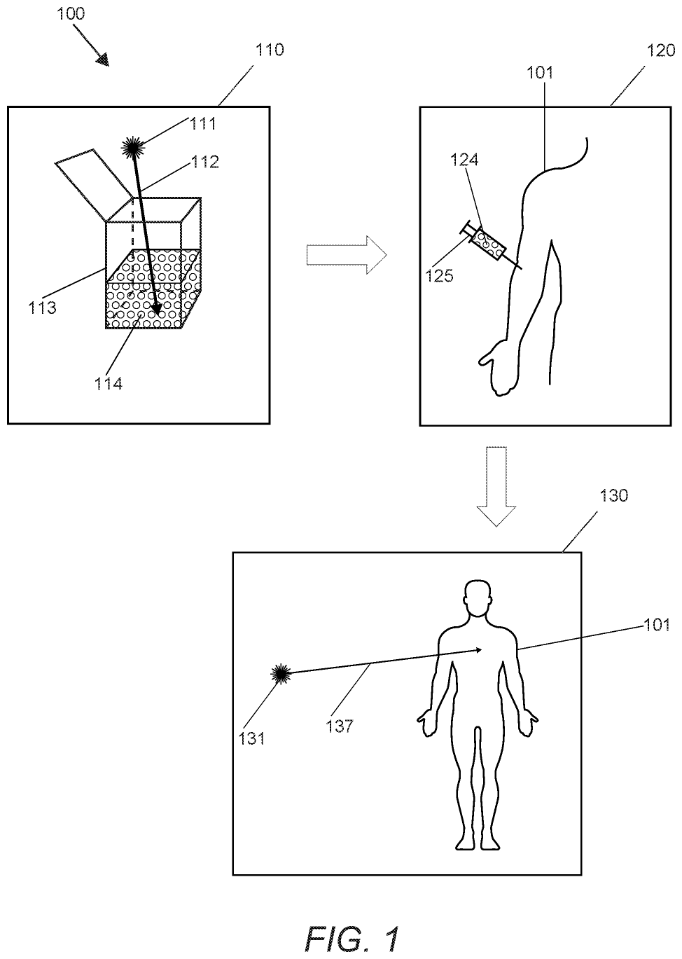

FIG. 1 is a flowchart 100 showing steps of (110) priming stem cells ex vivo with a laser beam, (120) infusing the primed stem cell into a patient, and (130) directing the primed stem cells to a target region of the patient. In Step 110, a laser source 111 is used to emit a laser beam 112 into a container 113 containing stem cells 114 to be primed. In Step 120, the primed stem cells 124 are injected intravenously into a patient 101 using a syringe 125. In Step 130, a laser source 131 is used to emit a laser beam 137 directed toward the patient 101, at a target region that needs healing (e.g., heart). Laser beam 137 and laser beam 112 share at least one matching energy signature, for example, frequency, intensity, waveform, and amplitude, etc. Preferably, laser beam 137 has the same frequency as laser beam 112, but is stronger in intensity than laser beam 112.

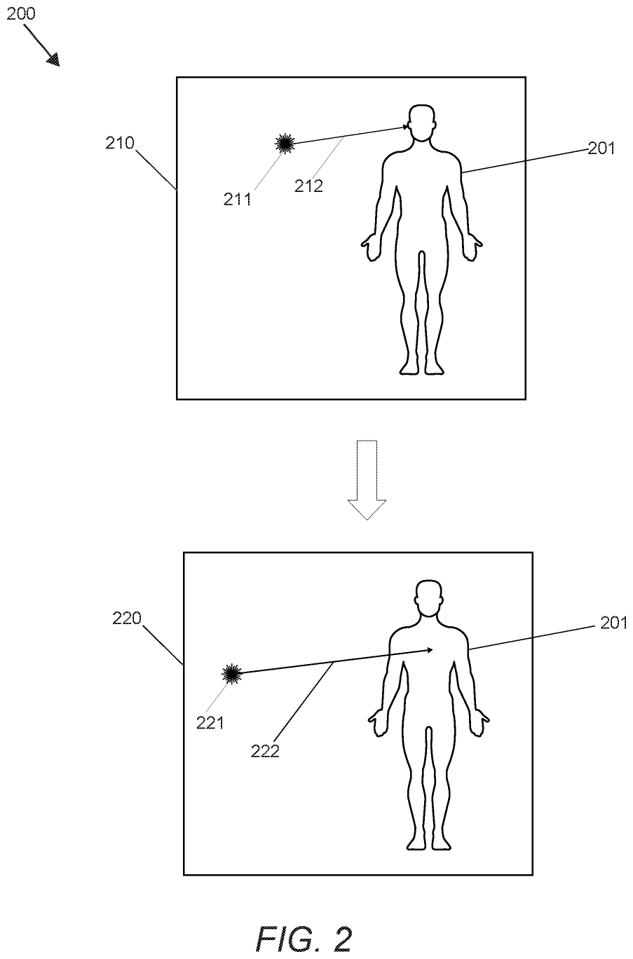

FIG. 2 is a flowchart 200 showing steps of (210) priming stem cells in vivo with a laser beam, and (220) directing the primed stem cells to a target region of the body. In Step 210, a laser source 211 is used to emit a laser beam 212 directed toward a patient 201, at a region where blood vessels are relatively close to the skin, for example, a mucus membrane (e.g., tympanic membrane). In Step 220, a laser source 221 is used to emit a laser beam 222 directed toward the patient 201, at a target region that needs healing (e.g., heart). Laser beam 222 and laser beam 212 share at least one matching energy signature, for example, frequency, intensity, waveform, and amplitude, etc. Preferably, laser beam 222 has the same frequency as laser beam 212, but is stronger in intensity than laser beam 212.

### EXAMPLE 1—HEART FAILURE STUDY

Typical stem cell therapy may require 3-6 weeks to observe results, which may be limited or not statistically significant. According to a meta-analysis of 1494 stage 4 heart failure patients who had umbilical stem cells inserted directly into coronary arteries via coronary catheterization, there was a 2.5% increase in ejection fraction after 90 days which represented an 8% increase in cardiac function. This improvement was found to be statistically insignificant.

In this study, 12 patients with stage 4 heart failure, awaiting heart transplant, received energy in the form of VSEL Laser to activate and guide VSELs. Unexpectedly, VSEL Laser primed and guided stem cells have frequently resulted in significant clinical improvement in patients in extremely short period time, sometimes within 24 hours. Also unexpectedly, in the VSEL treated group, there was a 3% improvement in ejection fraction and 8% improvement in overall cardiac function in just three days. Surprisingly, there was a 50% improvement in cardiac ejection fraction after 90 days. The results seen at 3 days and at 90 days were both highly statistically significant at p<0.01 and p<0.001, respectively. All 12 patients in the study were able to be removed from the heart transplant list.

### EXAMPLE 2

The laser guided stem cell study was conducted in Eastern Europe with 10 men with severe end stage heart failure. A normal heart pumps out 50-65% of the blood in the ventricles in the resting state. This percentage is called the cardiac ejection fraction (EF). When this falls below 50%, heart failure is said to be present. When the EF is less than 30%, severe heart failure is defined. The persons in this study had severe end stage disease with an average ejection fraction of 21%, and were considered candidates for heart transplantation.

The study used cord blood stem cells. These cells are derived from the blood in the umbilical cord of healthy newborn babies. After informed consent from each patient, the cells were primed with the laser for this purpose and then infused IV. Then a laser guidance process was used directing the beam toward the heart from the anterior chest and left lateral chest to give the strongest signals for localization to the heart muscle.

Most research protocols in this area wait 3-6 months before doing follow up studies as this is the time usually required to see a response. However, in this study, by the third day the average improvement in heart function was 14%. This increased further to about 25% in one month, 37% in 2 months and 50% in 3 months. Half of the persons improved off the heart transplant list and 20% to nearly normal after a single application. All the patients had improved function after 3 months. The least improved patient was 15% better. The greatest increase in function was 115% from an ejection fraction of 20% to 43%, or an enhancement of function nearly to a normal level.

A tissue repair index is a measure of the amount of change divided by the number of days since the treatment. The cardiac repair index is the percentage change in heart function ((new EF−original EF/original EF)×100%) divided by the number of days since the intervention. At 3 days post laser guided stem cells the cardiac repair index was 4.8% per day. For cells infused directly into the heart, the measured cardiac repair index at 6 months was 0.044% per day. The expected result was that intra-cardiac cells would work 10 times better. Unexpectedly, the laser guided cells given IV achieved a cardiac repair index that was over 100 times greater than direct intra-cardiac delivery. This suggests that both the rate and degree of regeneration can be increased with laser guidance.

### EXAMPLE 3—ANTI-AGING

For the general anti-aging protocol, 3 blue top tubes of blood are drawn from a vein. If a joint injection is also to be done, an additional tube of blood is obtained from a vein. The blood is then processed to yield a preparation of plasma, platelets, growth factors, dormant stem cells, and adult stem cells (fewer in number and tend to be less potent with age). The red cells and other potentially inflammatory cells are nearly completely removed. This preparation is then primed with laser in particular to awaken the dormant cells and potentially make the adult stems cells more active as well. The longest interval from drawing blood to delivering the cells is 4 hours. Durations shorter than this can be used also with good results. In general the autologous blood product is infused intravenously. In some cases, the preparation is also injected with a needle into an affected joint. The laser guidance process ideally begins within 5 minutes of infusing the autologous stem cells and growth factors. Depending on the focus, the typical duration is 5-15 minutes with laser applied to the target areas through the skin in order of highest tissue priorities. The laser beam is red and of low energy known to be safe for applying to the body. The power level is so low that the skin is barely warmed, far too low to cause pain or burning. Once the laser guidance step has been done the procedure is complete.

### EXAMPLE 4

For cardiac, neurological and joint conditions we have seen sequential improvement with allowing the benefits of one procedure to plateau and then doing a subsequent treatment. This can be in increments that vary from time to time and procedure to procedure. In general the focus is to get as much benefit from a given intervention and for that to suffice.

A good example of this phenomenon can be seen with chronic heart failure. The more advanced the condition the more it may be necessary to get an incremental boost with each procedure and space them closely enough that the result is an increasing level of function that builds to the highest achievable plateau. Then in some circumstances this may require periodic procedures to maintain the peak level of benefit.

### OTHER EXAMPLES

One patient with a stroke for 6 years had the fingers of his right hand curled into a claw without sensation or function. Within one hour of treating the patient with techniques of the current application, he could move and feel his fingertips. By the second hour he could play the piano again, including Mozart's “Midnight Sonata.”

As an example of stepwise benefits, a man with a mid-thoracic spinal injury was seen who had no sensation or motor function below his mid-chest and mid-upper back. After the first treatment he regained sensation about 6 inches down his chest and back and had recovery of erectile function. His second procedure 6 weeks later brought feeling down to his toes. The third procedure, about 6 months after the first, gave postural stability to stand after about 3 months. Three months later he was able to rise out of his wheelchair under his own power and walk the length of a football field albeit using a balance support device.

A woman with Parkinson's progressive over 20 years had a shuffling gait with short steps and could not walk heel to toe. One hour after treating her with techniques of the current application, she had a longer smoother stride with better arm swing and could walk heel to toe across the floor. Her ability to stand on one foot increased from 3-5 seconds to at least 10-15 seconds.

A man with multiple sclerosis progressive over 3 years had weakness and spastic muscle tone of his left leg and arm with little sensation below his left elbow or knee. He had to use his hands to lift his left leg off of the chair. 90 minutes after treating him with techniques of the current application, he could lift his left leg easily for the first time in over a year. He also had a return of significant sensation in his left lower leg and arm.

A man with quadriplegia with no sensation and limited motor function from his uppermost chest and upper arm down had no change in his function for 19 years. Five hours after treating him with techniques of the current application, he could feel down his arms to his elbows. One day later he could feel down to his wrists with sensation returning 2-3 inches down his front and back as well. Over about 3 months he noted increased sensations in his chest, feeling in his thumbs, and building his muscle mass and coordination in his arms.

A woman with bone on bone right hip joint disease with moderate pain and restriction of motion opted for the laser guided procedure that included joint injection with the cells. 90 minutes after treating her with techniques of the current application, flexion of her hip increased from 40 degrees to 95-100 degrees, and internal and external rotation increased from about 15-20 degrees each to 45 and 60 degrees, respectively, nearly into the normal range. The pain in the back of the hip was reduced 80% and in the front of the hip 100%.

As yet another example, a world class surfer suffered a severe ACL tear of his left knee. His ACL was hanging on by a thread and surgery was contemplated. The laser guided protocol was done with cells delivered IV and into the joint. The repair was rapid and complete. His return to competitive big wave surfing was accelerated and surgery was not required.

What is claimed is:

1. An ex vivo method of relocating stem cells to a target region of a patient, comprising:
priming the stem cells by subjecting the stem cells to a first energy field having a first energy signature;
intravenously infusing the primed stem cells into the patient; and
directing the primed stem cells to a target region by directing a second energy field to the target region, wherein the second energy field has a second energy signature that matches the first energy signature of the first energy field.

2. The method in claim 1, wherein the first energy signature comprises a first frequency, the second energy signature comprises a second frequency that is within 1000 Hz variation from the first frequency.

3. The method in claim 2, wherein the second frequency is within 100 Hz variation from the first frequency.

4. The method in claim 3, wherein the second frequency is within 10 Hz variation from the first frequency.

5. The method in claim 4, wherein the second frequency is within 1 Hz variation from the first frequency.

6. The method in claim 1, further comprises chemically priming the stem cells by exposing the stem cells to a growth factor.

7. The method in claim 6, wherein the growth factor is selected from the group consisting of fibroblast growth factor (FGF), insulin, insulin-like growth factors, vascular endothelial growth factor (VEGF), and migration-stimulating factor.

8. The method in claim 6, wherein the step of chemically priming the stem cells is performed before priming the stem cells by subjecting the stem cells to the first energy field.

9. The method in claim 6, wherein the step of chemically priming the stem cells is performed simultaneously with priming the stem cells by subjecting the stem cells to the first energy field.

10. The method in claim 6, wherein the step of chemically priming the stem cells is performed after priming the stem cells by subjecting the stem cells to the first energy field.

11. An in vivo method of relocating stem cells to a target region of a patient, comprising:
subjecting at least a portion of the body of the patient to a laser configured to prime the stem cells, the first energy field having a first energy signature; and
directing the primed stem cells to a target region of the body by directing a second energy field into a portion of the body, wherein the second energy field has a second energy signature that matches the first energy signature of the first energy field.

12. The method in claim 11, wherein the first energy signature is a first frequency, the second energy signature is a second frequency that is within 1000 Hz variation from the first frequency.

13. The method in claim 12, wherein the second frequency is within 100 Hz variation from the first frequency.

14. The method in claim 13, wherein the second frequency is within 10 Hz variation from the first frequency.

15. The method in claim 14, wherein the second frequency is within 1 Hz variation from the first frequency.

16. The method in claim 11, wherein a portion of a mucous membrane of the patient is subjected to the first energy field.

17. The method in claim 11, wherein the patient's entire body is subjected to the first energy field.

18. The method in claim 11, wherein the target region comprises at least one of brain, spinal cord, and heart.

19. A method of treating a disease, using the method described in claim 1, wherein the disease is selected from the group consisting of heart disease, spinal cord injury, and neurodegenerative disease.

20. A method of treating a disease, using the method described in claim 11, wherein the disease is selected from the group consisting of heart disease, spinal cord injury, and neurodegenerative disease.
